(19) United States
(12) Patent Application Publication
(10) Pub. No.: US 2025/0197926 A1
(43) Pub. Date: Jun. 19, 2025

(54) SYSTEM FOR DISINFECTING TEXTILES IN A HOUSEHOLD APPLIANCE

(71) Applicant: CONTROLADORA MABE, S.A DE C.V., Ciudad de México (MX)

(72) Inventors: Juan Roberto CAPETILLO GONZÁLEZ, Querétaro (MX); Gustavo PONCE MARTINEZ, Querétaro (MX); Carlos Alberto RICARDEZ MACIAS, Querétaro (MX); Victor Hugo MIRANDA RAZO, Querétaro (MX); Gabriel ZALDIVAR BALVANERA, Querétaro (MX); Carolina TREJO VÁZQUEZ, Querétaro (MX)

(21) Appl. No.: 18/971,390

(22) Filed: Dec. 6, 2024

Related U.S. Application Data
(63) Continuation of application No. 17/821,950, filed on Aug. 24, 2022.
Application No. 17/821,950 is a continuation of application No. 15/906,964, filed on Feb. 27, 2018, now Pat. No. 11,459,606.
Application No. 15/906,964 is a continuation of application No. 15/390,806, filed on Dec. 27, 2016, now abandoned.
Application No. 15/390,806 is a continuation of application No. 13/147,159, filed on Nov. 15, 2011, now abandoned.
(60) Provisional application No. 61/148,737, filed on Jan. 30, 2009.

(30) Foreign Application Priority Data
Dec. 15, 2023 (MX) MX/A/2023/015414

(51) Int. Cl. C12Q 1/6869; C07H 21/04

(52) U.S. Cl. CPC C12Q 1/6869; C07H 21/04

(57) ABSTRACT

The present invention relates to a system for disinfecting textiles with UV technology comprising an ultraviolet light assembly comprising a power supply; a housing to contain the light assembly and protect it from any impact; an electronic card that is responsible for switching the ultraviolet light assembly on or off. The system allows disinfecting textiles by up to 99.9%, saving energy and, at the same time, reducing process time and damage to the textiles.

### OBJECT OF THE INVENTION

The present invention refers to a UV technology-assisted system for disinfecting textiles in a household appliance that allows up to 99.9% disinfection of textiles, saving energy and, at the same time, reducing process time and damage to textiles.

### BACKGROUND

The use of UV technology for textile disinfection is known in the prior art, such as in AU 2014393062, which refers to a laundry washing machine comprising an external washing tub, a rotating washing drum adapted to receive laundry, and a recirculation circuit comprising a UV source within said recirculation circuit. Said recirculation duct comprises a duct for said liquid to flow through and a seat for receiving said UV source, including a UV-transparent wall, wherein a portion of the UV-transparent wall separates said seat from the duct.

Document KR 100730936 is also known, which describes a drum washing machine that has a sterilizing and deodorizing function capable of disinfecting any microorganisms and bacteria that remain in the laundry during the laundry drying operation and eliminating the odors generated in the drum by residual contaminants. The washing machine is composed of a cabinet, a water storage tank installed horizontally from the top of the base, a drum rotatably installed in the water storage tank, a door, as well as two ultraviolet generating units installed on the washing machine door to radiate ultraviolet rays into the drum.

Another known document is KR 101565953, which refers to an apparatus for the disinfection of laundry using UV LEDs, the invention comprising: a cabin for housing the laundry inside and fitted with a door on one surface; a respective UV LED lamp capable of radiating UV light having a wavelength with a disinfection function; and a hanger unit configured to work in conjunction with the lamp. When laundry is hung on the hanger unit, the push button is pressed and a hanger signal is transmitted to the control unit so that the UV LED lamp turns on, and when the laundry is removed from the hanger unit, the push button is controlled to return to its original position, transmitting a signal to the control unit controlling the UV LED lamp to turn off.

Document U.S. Pat. No. 5,664,340 protects a dryer designed to remove fungus-causing bacteria and germs in laundry. The present invention consists of ultraviolet mechanisms located inside the door of typical gas and electric dryers, thus eliminating germs from washed laundry. The UV lights are contained within a plate mounted on the dryer door and covered with transparent glass or polycarbonate plastic. When the dryer is on, the ultraviolet lights will shine, thus starting the process of germ removal from the laundry.

Document CN 106012406 describes a washing machine with ultraviolet radiation for a sterilization and disinfection function that includes a washing machine body, an inner tub placed in the washing machine body, the bottom of the inner wall of the inner tub is additionally provided with an impeller, wherein a coil is provided in the impeller, the coil is electrically connected to an ultraviolet lamp which is fixedly installed in the impeller, and a magnetic field generator to act on the connecting coils and generate electric current is provided in the inner tub. When the coils generate current, power is supplied to the lamp, which generates ultraviolet rays and irradiates the laundry to be washed inside the inner tub.

Despite these prior efforts, there is still a need for effective alternatives to existing systems for disinfecting textiles that allow their use in different embodiments and provide additional benefits such as energy savings, shorter process times and less damage to textiles by avoiding using high temperatures for disinfection.

### BRIEF DESCRIPTION OF THE INVENTION

The present invention relates to a system for disinfecting textiles in a household appliance, comprising: a housing arranged inside the household appliance to house an ultraviolet light assembly; an electrical power supply for operating the ultraviolet light assembly; said ultraviolet light assembly points to the interior of a drum of the household appliance, said ultraviolet light being angularly guided with respect to a horizontal plane; and an electronic card in communication with the power supply of the ultraviolet light assembly to control its activation/deactivation.

The description provides illustrative examples of various aspects and embodiments of the present invention, and an overview or framework is intended to be provided for understanding the nature and character of the aspects and embodiments claimed. The present invention may present several modifications and alternative constructions, some of which are detailed in the drawings below. However, it should be clear that the intention is not meant to limit the invention to a particular embodiment or form, but rather the present invention should cover changes, additions and modifications as part of its scope. Independent aspects and advantages of the present invention will become apparent to those skilled in the art upon review of the detailed description and figures.

The accompanying figures are included to provide further illustration and understanding of the various aspects and embodiments, and are incorporated into and constitute a part of this specification. The Figures, together with the specification, serve to explain the aspects and embodiments described and claimed.

### BRIEF DESCRIPTION OF THE DRAWINGS

The illustrative embodiment can be described with reference to the accompanying figures, whereby:

FIG. 1 illustrates a rear section view showing the UV technology-based system for disinfecting textiles applied in a household appliance.

FIG. 2 illustrates a lateral section view of the UV technology-based system for disinfecting textiles applied in a household appliance.

FIG. 3 illustrates an ultraviolet light assembly of the present invention.

FIG. 4 illustrates the ultraviolet light assembly of the present invention in exploded view.

FIG. 5 illustrates the ultraviolet light assembly of the present invention in exploded view assembled in a household appliance.

### DETAILED DESCRIPTION OF THE INVENTION

The following detailed description is exemplary only and is not intended to limit the described embodiments or the application and uses of the described embodiments. As used herein, the word “exemplary” or “illustrative” means “serving as an example, instance, or illustration”. Any implementation described herein as “exemplary” or “illustrative” should not necessarily be construed as preferred or advantageous over other implementations. All of the implementations described below are exemplary implementations provided to enable those skilled in the art to make or use the embodiments of the disclosure and are not intended to limit the scope of the disclosure. In addition, for purposes of description herein, the terms “end”, “upper”, “lower”, “right”, “left”, “vertical”, “horizontal”, “lateral”, “longitudinal” and derivatives thereof are related to the invention as shown in the figures. In addition, there is no intention to be subject to any explicit or implicit theory presented in the technical field above, background, brief summary or the following detailed description. It should also be understood that the specific devices and processes illustrated in the accompanying drawings, and described in the following description, are merely exemplary embodiments of the inventive concepts defined in the attached claims.

The illustrations generally show non-limiting aspects of the systems and methods of the present description. The various aspects of the present descriptions of the devices should not be construed in any way as limiting the description. In addition, any modifications, concepts, and applications of aspects of the description are to be construed by those skilled in the art as comprised by, but not limited to, the illustrations and descriptions herein. Several modifications, equivalents, variants and alternatives, however, will be readily apparent to those skilled in the art. Any and all such modifications, variants, equivalents and alternatives are intended to fall within the spirit and scope of the present description.

As used herein, the singular articles “a”, “an”, and “the” include plural referents unless expressly and unambiguously limited to one referent. The term “proximal” refers to a direction toward the center or a central region of a device. The term “distal” refers to an outward direction extending away from a central region of a device. However, it is to be understood that the description may assume several alternatives, variations, and sequences of steps, except where expressly stated otherwise. It is also to be understood that the specific devices and processes illustrated in the accompanying drawings and described in the following description are non-limiting representations of several aspects of the description. Therefore, the specific dimensions and other physical characteristics related to the aspects described herein are not intended to be and should not be considered as limiting.

Unless otherwise indicated, all ranges or ratios described herein are meant to encompass any and all sub-ranges or sub-ratios incorporated herein. For example, an established range or ratio of “1 to 10” should be considered to include any and all sub-ranges between (and including) the minimum value of 1 and the maximum value of 10; that is, all sub-ranges or sub-ratios that start with a minimum value of 10 or less, such as, but not limited to, 1 to 6.1, 3.5 to 7.8, and 5.5 to 10. Unless otherwise indicated, all numbers expressing dimensions, quantities of ingredients, flow rates, pressures, etc., used in the description and claims, are to be construed as modified in all instances by the term “approximately”. For the purposes of the present description, the term “approximately” determines a range of +10%, whereby if a value of “approximately 30%” is specified, the value is within a range of 27% to 33%.

The term “a plurality” used throughout this description refers to a defined number of elements that ranges from 2 to 20.

The term “at least” used throughout this description indicates a minimum value or lower limit for the element to which it refers.

All documents, such as but not limited to issued patents and patent applications, referred to herein, unless otherwise indicated, are to be deemed to be “incorporated by reference” in their entirety.

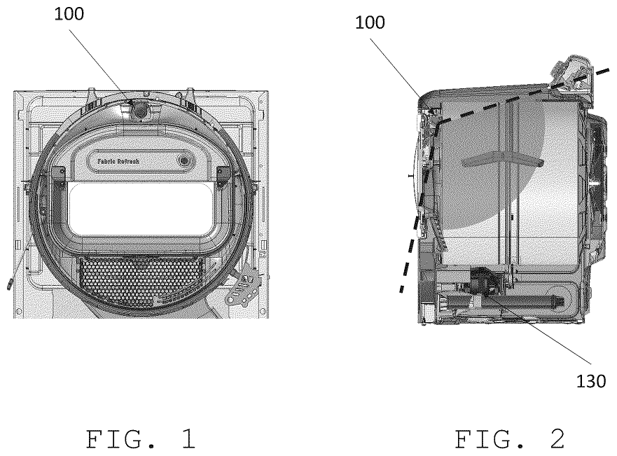

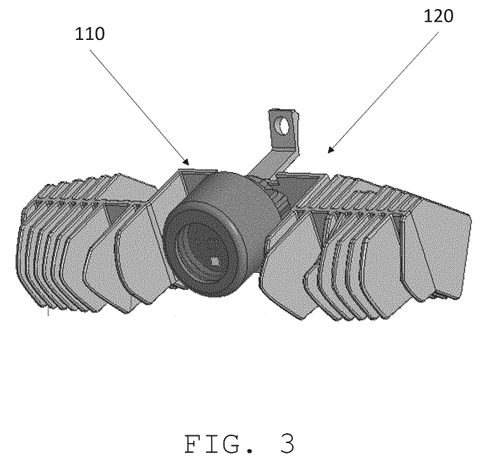

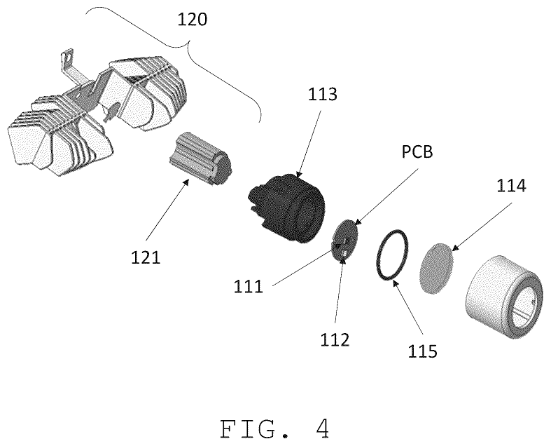

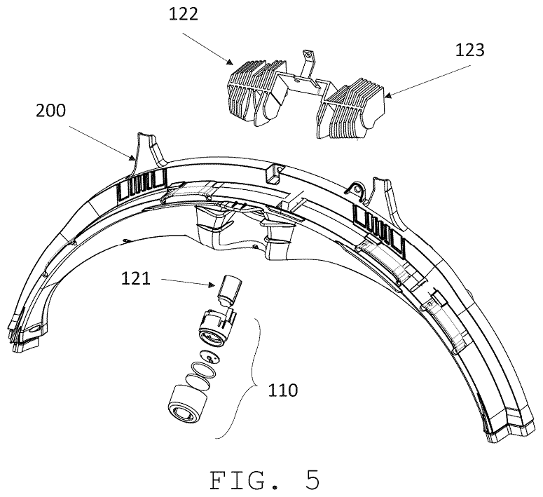

The present invention relates to a system for disinfecting textiles in a household appliance (100) comprising an ultraviolet light assembly (110) that is activated by a power supply. In a preferred embodiment, the power supply comprises electronic means. Preferably, the power supply is selected from one of a driver and a source, preferably a driver. Said ultraviolet light assembly (110) is configured to point into the interior of a drum of the household appliance. In the illustrated embodiment, said ultraviolet light assembly (110) is angularly guided with respect to a horizontal plane, such that said ultraviolet light assembly (110) is installed in a housing (200) arranged inside the household appliance, so that it can directly radiate to the textiles and to the internal surfaces of the household appliance; preferably said housing is located in the upper bearing of the drum of the household appliance (FIGS. 1, 2 and 5); in an alternative embodiment, the ultraviolet light assembly is angled with respect to said horizontal plane.

In one embodiment of the invention, the ultraviolet light assembly (110) is selected from the group comprising at least one UV light LED (111) and at least one visible light LED (112) mounted on a printed circuit board (PCB) inside a case (113).

In one embodiment, said at least one UV light LED (111) and at least one visible light LED (112) are activated independently; in an alternative embodiment, said at least one UV light LED (111) and at least one visible light LED (112) are activated simultaneously.

Said ultraviolet light assembly (110) has the function of radiating ultraviolet light onto the textiles inside the drum and to the internal components of the household appliance, for example, the drum, baffles, air diffuser and suction duct to eliminate up to 99.9% of viruses and bacteria, reducing the amount of microorganisms present in the textiles and preventing their growth inside the household appliance, thus preventing them from being transferred to the textiles to be placed inside in the next cycle.

In one embodiment of the invention, the case (113) has a quartz lens (114) in front of said at least one UV light LED (111) and at least one visible light LED (112); said quartz lens (114) has a finish selected from one of a frosted finish and a sand blast finish; in an alternative embodiment, an O-ring seal (115) is arranged between the quartz lens (114) and the case (113) to prevent moisture from entering the interior of the case (113).

In an alternative embodiment, the UV light assembly (110) has at least one heat sink (120) that may be composed of one or more individual parts that are in contact with each other. In one embodiment, the at least one heat sink is a monolithic piece; in an alternative embodiment, the at least one heat sink has at least one heat sink extension (121), the at least one heat sink extension (121) being in direct contact with the at least one heat sink; in a further embodiment, the case (113) is mounted on the at least one heat sink extension (121); said at least one heat sink comprises a first and a second section (122, 123) of metal plates side by side, said metal plates being arranged parallel to each other.

In a preferred embodiment of the invention, the ultraviolet light is chosen from UVC, UVA, UVB and UVV to allow effective elimination of microorganisms from textiles, enhance the whitening of clothes, preserve the fragrance of fabric softener after the washing process, reduce damage to clothes (wrinkling, shrinkage), preserve color, among others. Therefore, its wavelength ranges from about 100 nm to about 400 nm.

The invention also comprises an electronic card responsible for controlling switching the ultraviolet light assembly (110) on or off, depending on the embodiment in which the present invention is used, as will be detailed later. Said electronic card is also responsible for switching on/off a driving means (130) that activates the movement of the drum and a heat generating element, said heat generating element being chosen from one of an electric resistance or a gas system.

The system of the present invention is intended to be applied in a dryer, preferably an open cycle dryer, as well as in a dryer with a heat pump or with a condensing system.

The present invention also provides a UV technology-based method for disinfecting textiles, said method being able to have different embodiments, as detailed below.

In a first embodiment, the UV light disinfection system of the present invention can be applied in any drying cycle, activating or deactivating the ultraviolet light assembly (110) by means of the electronic card at the convenience of the user. In this embodiment, the ultraviolet light assembly (110) is on 100% of the time.

In a second embodiment, the system is applied in a special disinfection cycle comprising stopping the movement of the drum and turning on the ultraviolet light assembly (110) at controlled time intervals; that is, prior to the end of a drying cycle; the electronic card deactivates the driving means (130) of the drum, stopping its movement so that the ultraviolet light assembly (110) irradiates all the components of the household appliance and the textiles inside it. This behavior (stopping the movement of the drum and irradiating all the components of the household appliance together with the textiles with ultraviolet light) can be repeated as many times as necessary.

As an example, a possible disinfection cycle for the interior of the household appliance for a drying process with a total duration of 1.5 hours is described below.
Pause #1:1-minute pause (deactivation of the driving means) and radiation of UV light
Drum rotation for 13 seconds
Pause #2:1-minute pause (deactivation of the driving means) and radiation of UV light
Drum rotation for 13 seconds
Pause #3:1-minute pause (deactivation of the driving means) and radiation of UV light
Drum rotation for 13 seconds

As the person skilled in the art will understand, the number of pauses and rotations are parametric, so they can be adjusted according to the needs of each case.

In a third embodiment, UV light and heat are applied alternately. The duration and number of applications between one technology and another will be defined by the person skilled in the art, according to the specific needs of each case.

The method and system of the present invention allow to efficiently reduce the amount of microorganisms in the textiles, as well as to reduce the time of the drying cycle (up to 50%), since disinfection is carried out within the same drying cycle, which also allows for energy savings and less damage to the textiles by reducing the process time and the use of high temperatures. In addition, the proposed method provides a flexible alternative to existing methods, since it allows its application in different embodiments, at the user's convenience.

A person skilled in the art can modify the structure described herein. However, it should be noted that this description relates to preferred embodiments of the invention, and is provided for illustrative purposes only, and should not be understood as limiting the invention. All obvious modifications in the spirit of the invention, such as changes in the shape, material, and dimensions of the elements that make up the invention, should be considered within the scope of the attached claims.

The invention has been described in an illustrative manner, and it should be understood that the terminology used herein is intended to correspond to the nature of the words of the description rather than to provide a limitation.

Obviously, many modifications and variations of the present invention are possible in light of the above teachings. Therefore, it should be understood that within the scope of the invention described, the invention may be practiced otherwise than specifically described.

1. System for disinfecting textiles in a household appliance comprising:
a housing arranged inside the household appliance to house an ultraviolet light assembly;
an electrical power supply to drive the ultraviolet light assembly;
said ultraviolet light assembly points to the interior of a drum of the household appliance, said ultraviolet light being angularly guided relative to a horizontal plane; and
an electronic card in communication with the power supply of the ultraviolet light assembly to control activation/deactivation thereof.

2. The system for disinfecting textiles in a household appliance of claim 1, wherein the ultraviolet light assembly comprises at least one UV light LED and at least one visible light LED mounted on a printed circuit board (PCB) within a case.

3. The system for disinfecting textiles in a household appliance of claim 2, wherein said at least one UV light LED and at least one visible light LED are activated one of independently or simultaneously.

4. (canceled)

5. The system for disinfecting textiles in a household appliance of claim 2, wherein the UV light LED that irradiates said ultraviolet light assembly has a wavelength ranging from about 100 nm to about 400 nm.

6. The system for disinfecting textiles in a household appliance of claim 2, wherein in front of said at least one UV light LED and at least one visible light LED there is a quartz lens and a case.

7. The system for disinfecting textiles in a household appliance of claim 6, wherein said quartz lens has one of a frosted finish or a sand blast finish.

8. (canceled)

9. The system for disinfecting textiles in a household appliance of claim 1, wherein the ultraviolet light assembly has at least one heat sink, wherein the at least one heat sink is a monolithic piece, wherein the at least one heat sink has at least one heat sink extension, wherein the at least one heat sink extension being in direct contact with the at least one heat sink, wherein said at least one heat sink comprises a first and a second section of metal plates side by side, said metal plates being arranged parallel to each other.

10. (canceled)

11. (canceled)

12. The system for disinfecting textiles in a household appliance of claim 2, wherein the case is mounted on the at least one heat sink extension.

13. The system for disinfecting textiles in a household appliance of claim 1, wherein the housing of the system is arranged in any component that allows light to penetrate into the drum, wherein the housing of the system is arranged in an upper bearing of the drum of the household appliance.

14. (canceled)

15. The system for disinfecting textiles in a household appliance of claim 7, wherein a seal is arranged between the quartz lens and the case.

16. (canceled)

17. The system for disinfecting textiles in a household appliance of claim 1, wherein it has a heat generating element.

18. A method for disinfecting textiles in a household appliance comprising:
activating the ultraviolet light assembly of the system of claim 1, for a certain time to irradiate the textiles and internal components of said household appliance;
where the ultraviolet light assembly is switched on by means of the electronic card.

19. The method for disinfecting textiles in a household appliance according to claim 18, wherein the wavelength of the ultraviolet light radiated by the ultraviolet light assembly is in the range of about 100 nm to about 400 nm.

20. The method for disinfecting textiles in a household appliance of claim 18, wherein the ultraviolet light assembly is switched on while a drum of the household appliance is in motion.

21. The method for disinfecting textiles in a household appliance according to claim 20, further comprising:
stopping the movement of the drum for a certain time before activating the ultraviolet light assembly; and,
repeating all the steps as many times as necessary.

22. The method for disinfecting textiles in a household appliance of claim 20, wherein the time the drum stops is about 1 minute and wherein the time during which the ultraviolet light assembly is activated is about 13 seconds.

23. The method for disinfecting textiles in a household appliance of claim 22, further comprising alternately applying ultraviolet light and heat by means of a heat generating element.

24. A household appliance comprising:
a drum
a driving means that activates the movement of the drum of the household appliance; and,
the system for disinfecting textiles of claim 1.

25. The household appliance of claim 24, wherein the household appliance is selected from one of a dryer, a washing machine and a washer-dryer, wherein the dryer is selected from a dryer with a heat pump and a dryer with a condensing system, wherein the dryer is an open cycle dryer.

26. (canceled)

27. (canceled)
